(19) United States
(12) Patent Application Publication
(10) Pub. No.: US 2003/0039657 A1
(43) Pub. Date: Feb. 27, 2003

(54) Inducible vaccines

(76) Inventors: Stephen Albert Johnston, Dallas, TX (US); Baoxi Qu, Plano, TX (US)

Correspondence Address: Fulbright & Jaworski LLP, Mark B Wilson, 600 Congress Avenue Suite 2400, Austin, TX, 78701, US

(21) Appl. No.: 09/549,903

(22) Filed: Apr. 14, 2000

Related U.S. Application Data
(60) Provisional application No. 60/129,597, filed on Apr. 15, 1999.

(51) Int. Cl. A61K 48/00; A61K 39/00

(52) U.S. Cl. 424/184.1; 424/93.21

(57) ABSTRACT

The present invention relates to methods of immunization by introducing DNA expression systems into a vertebrate species. Such DNA expression systems preferably include DNA sequences encoding polypeptides of pathogens and antigens and an additional DNA sequence encoding a regulatory moiety which can bind with an administered activating agent to induce expression of an immune response. The present invention also relates to methods of administration of DNA expression systems into the species. The methods of this invention are useful for prophylactic vaccination or therapeutic immunization against infectious disease causing pathogens and other tumor/cancer antigens.

The present application claims the priority of co-pending U.S. Provisional Patent Application Serial No. 60/129,597, filed Apr. 15, 1999, the entire disclosure of which is incorporated herein by reference without disclaimer. The government may owns rights in the present invention pursuant to a grant from the Defense Advanced Research Projects Agency.

### BACKGROUND OF THE INVENTION

1. Field of the Invention

The present invention relates generally to the fields of DNA-based vaccines. More particularly, it concerns development of inducible vaccines wherein antigen levels can be induced by administration of an activating agent (regulatory moiety) such as a small molecule or drug at any point of time in the animal.

2. Description of Related Art

Viral, bacterial and pathogenic diseases in animals including humans are the major problem. Owing to the high density of animals in the farm areas, infectious diseases may adversely affect or even eradicate a large proportion of the stock in, for example, pigs, cattle, sheep, chickens, turkeys, horses etc. Prevention of disease is a more desired remedy to these threats than intervention once the disease is in progress. Vaccination of animals or humans is the only preventative method which may offer long-term protection through immunity. For humans vaccination may also be the preventive remedy to diseases such as cancers and malignant tumors wherein immune responses can be artificially stimulated to tumor markers thereby eliminating the tumor cells.

Animals and humans have been immunized by antigen-based immunization methods using live attenuated pathogens, killed whole pathogens, sera drawn from other animals or more recently, in laboratory settings, by recombinant proteins. While live attenuated vaccines induce good humoral and cell-mediated immune responses and can be administered orally or by injection, there is the important risk of reversion to a virulent form. Whole live attenuated vaccines are not preferred in industrial farming due to the risk of contaminating other species—a live attenuated vaccine which may be generally safe for the target species may be virulent in other species of animals.

Vaccines using whole killed bacteria (i.e. bacterins) or recombinant proteins from pathogens expressed in cell lines (subunit vaccines) have the disadvantage of inducing short-lived immune responses. Injected antigen, including recombinant protein, is processed solely in an exogenous form usually causing induction of the humoral response (i.e. production of antibodies, Th1) but often a failure to induce cell-mediated immunity (i.e. cytotoxic T-cells, Th2).

Another disadvantage of whole killed and subunit vaccines is that they almost always must be injected and they require an adjuvant to induce an effective immune response. Intramuscular injections of these adjuvants can cause granuloma formation which scars the flesh and lowers the market value of the animal and has undesirable side-effects on humans. Intraperitoneal injection of adjuvants may cause adhesions between the viscera which can affect the health of the animals.

Recombinant protein vaccines are difficult and expensive to make especially if the protein must be purified. For example, bacterially-expressed recombinant proteins may form inclusion bodies from which recovery of protein in correct configuration may be low or nonexistent. Induction of an immune response may require that the antigenic protein be correctly glycosylated and folded, which may not be accomplished in a cell other than an animal cell thereby requiring expression in specific eukaryotic expression systems.

Some of the current methodologies for administering vaccines are not technically or economically practical. For example, direct injection of recombinant and whole killed pathogen vaccines into farm animals is labor intensive and expensive relative to the future market value of the animal. Furthermore, injection needles can cross-infect animals with contaminating pathogenic organisms, and accidental injection of humans can cause severe or fatal infections and anaphylactic reactions. Moreover, non-injurious injection of small animals is very difficult, especially in young animals, which are particularly susceptible to disease.

A less expensive and easier method which has been used to administer killed viral or bacterial vaccines is an oral method wherein the vaccine is added directly to the water or incorporated into animals food. Oral vaccines have historically shown inconsistent and relatively low levels of protection suggesting that they may be best used as a method of re-vaccination.

Genes have been introduced directly into animals by using live viral vectors containing particular sequences from an adenovirus, an adeno-associated virus, or a retrovirus genome. The viral sequences allow the appropriate processing and packaging of a gene into a virion, which can be introduced to animals through invasive or non-invasive infection. Viral vectors have several disadvantages. Viral vectors being live pathogens, still carry the risk of inadvertent infection. Furthermore, proteins from viral vector sequences induce undesirable inflammatory or other immune responses which may prevent the possibility of using the same vector for a subsequent vaccine or boost. Viral vectors also limit the size of the target gene that can be expressed due to viral packaging constraints.

Naked DNA transfects relatively efficiently if injected into skeletal muscle but poorly or not at all if injected into other tissues (Wolff et al., 1990, incorporated herein by reference). Plasmid DNA coated onto the surface of small gold particles and introduced into the skin by a helium-driven particle accelerator or “gene-gun” can directly transfect cells of the epidermis and dermis (Pecorino and Lo, 1992, which is incorporated herein by reference).

DNA has also been introduced into animal cells by liposome-mediated gene transfer. DNA-liposome complexes, usually containing a mixture of cationic and neutral lipids, are injected into various tissues or instilled into the respiratory passages. Nabel et al., 1992, which is incorporated herein by reference, have shown that liposomes may be used to transfect a wide variety of cell types by intravenous injection in mammals. In addition, liposome-mediated gene transfer has been used to transfer the cystic fibrosis transmembrane conductance gene into the nasal epithelium of mice and humans suffering from cystic fibrosis (Yoshimura et al., 1992; Caplan et al.,1995, respectively, both of which are incorporated herein by reference).

Substances may also be administered using biodegradable microspheres composed of polymers such as polyester poly(lactide-co-glycolide) (Marx et al., 1993, incorporated herein by reference). It is notable that these particles can survive the upper digestive system and arrive intact in cells of gut-associated lymphoid tissue (Eldridge et al., 1989, incorporated herein by reference). Biodegradable microspheres have been used to deliver recombinant antigens, toxoids or attenuated virus into mammals by systemic and oral routes (O'Hagan et al., 1991; O'Hagen et al., 1993; Eldridge et al., 1991 incorporated herein by reference). They may also be useful to deliver recombinant plasmid DNA to gut-associated lymphoid tissue for the purpose of immunization.

Plasmid DNA encoding reporter genes have been successfully introduced into animals by intramuscular injection and can express proteins from a foreign gene with the vector constructs containing the basic elements (i.e. backbones, promoter and enhancer elements).

The induction of an immune response to a protein expressed from an introduced gene was first suggested by Acsadi et al., 1991, which is incorporated herein by reference, who found that after plasmid DNA transfer into rat cardiac muscle, reporter gene expression was transient but could be prolonged by treatment with an immunosuppressant. Subsequently, it was shown that antibodies were induced in rodents against human growth hormone (Tang et al., 1992; Eisenbraun et al., 1993, both of which are incorporated herein by reference) or human .alpha.-antitrypsin (Tang et al., 1992, also incorporated herein by reference) when these proteins were expressed from DNA coated onto gold particles and introduced into cells of the skin by bombardment.

DNA-based immunization refers to the induction of an immune response to an antigen expressed in vivo from a gene introduced into the animal. This method offers two major advantages over classical vaccination in which some form of the antigen itself is administered. First, the synthesis of antigen in a self-cell mimics in certain respects an infection and thus induces a complete immune response but carries absolutely no risk of infection. Second, foreign gene expression may continue for a sufficient length of time to induce strong and sustained immune responses without boost.

Several mammalian animal models of DNA-based immunization against specific viral, bacterial or parasitic diseases have been reported. These include influenza (Fynan et al., 1993; Montgomery et al., 1993; Robinson et al., 1993; Ulmer et al., 1993); HIV (Wang et al. 1993); hepatitis B (Davis et al., 1993); malaria (Sedagah et al., 1994); bovine herpes (Barry et al., 1995); mycoplasma (Cox et al., 1993); herpes simplex (Rousse et al., 1994; Manicken et al. 1995); rabies (Xiang et al., 1994); lymphocytic choriomeningitis (Yokoyama et al., 1995); and tuberculosis (Lowrie et al., 1994), all of which are incorporated herein by reference. In most of these studies a full-range of immune responses including antibodies, cytotoxic T lymphocytes (CTL), T-cell help and (where evaluation was possible) protection against challenge was obtained. In these studies naked DNA was introduced by intramuscular or intradermal injection with a needle and syringe or by instillation in the nasal passages, or the naked DNA was coated onto gold particles which were introduced by a particle accelerator into the skin.

There is a need for novel systems to vaccinate animals and humans against diseases. These systems should be inexpensive to produce and administer, avoid the use of live, attenuated organisms, and induce strong and long-lasting immunity preferably without boost and with induction of both antibodies and cell-mediated immunity. More preferably, the system should be applicable to small animals, be less stressful to animals during administration, and have the capacity of simultaneously immunizing many animals for reduced labor-related costs. Most importantly the method should allow for induction of the immune response by extrinsic means.

### SUMMARY OF THE INVENTION

The present invention relates to the immunization of animals and humans by DNA expression systems and overcomes many disadvantages associated with the existing antigen-based vaccines. The invention relates to introduction of DNA plasmids (alone or in a formulation) containing sequences encoding antigenic components of viral, bacterial or parasitic diseases by transfection into the animal species. The methods and compositions of this invention are useful for immunization (i.e. for prophylactic vaccination or therapeutic immunization) of animals and humans against infectious and non-infectious diseases. The present invention also envisions DNA plasmids (alone or in formulation) containing sequences encoding tumor/cancer marker proteins which can generate an immune response against cancers/tumors and provide cancer therapy. The DNA sequences according to this invention are preferably present in vectors capable of inducing protein expression of these sequences (i.e. expression vectors) and may be administered in combination with other DNA sequences in the same or other expression vectors or as oligonucleotides. In the most preferred embodiment, these additional DNA sequences may encode other regulatory proteins which are capable of inducing the expression of the antigen when the animal or human is administered with an activating agent or a regulatory moiety, e.g., a drug or a small molecule. In addition, there may be additional DNA elements encoding cytokines, co-stimulatory molecules, or may include immunostimulatory sequences (e.g., CpG motifs). The DNA sequences may also be given with other adjuvants, such as alum, mineral oil etc.

The invention describes compositions for inducing an immune response in an animal comprising: an expression vector containing a DNA sequence encoding at least one immunogenic polypeptide the expression vector further encoding an expression control sequence, under the control of a regulatory element, capable of directing expression the immunogenic polypeptide; at least one regulatory protein; and at least one regulatory moiety, wherein the expression of the immunogenic polypeptide is controlled by the regulatory protein wherein the regulatory protein interacts with the regulatory moiety and activates the regulatory element.

In another embodiment of the invention the regulatory protein is encoded by a second expression vector. In an additional embodiment of the invention the regulatory moiety interacts with the regulatory protein and renders it active. In a further embodiment of the invention the activated regulatory protein interacts with the regulatory element of the expression vector and directs expression of the immunogenic polypeptide.

In one embodiment of the invention the regulatory moiety is a small molecule. In another embodiment of the invention the regulatory moiety is a drug. In a further embodiment of the invention the regulatory moiety is administered orally. In yet another embodiment of the invention the regulatory moiety is administered intravenously.

In still another embodiment of the invention, an adjuvant is administered in addition to the regulatory moiety. In a further embodiment of the invention, the adjuvant is selected from a group consisting of alum, CpG oligonucleotides, guanosine tetraphosphate or guanosine pentaphosphate.

A further aspect of the invention contemplates a method for inducing an immune response in an animal which comprises administering: an expression vector containing a DNA sequence encoding at least one immunogenic polypeptide, the expression vector further encoding an expression control sequence, under the control of a regulatory element, capable of directing expression of the immunogenic polypeptide; and at least one regulatory moiety, wherein the expression of the immunogenic polypeptide is controlled by a regulatory protein wherein the regulatory protein further interacts with the regulatory moiety.

In one aspect of the method, the regulatory protein is endogenously encoded in the animal. In another aspect of the invention, the method further comprises administration of the regulatory protein. A related embodiment of the method comprises administering a second expression vector, wherein, the regulatory protein is encoded by the second expression vector.

In a further aspect of the invention the regulatory moiety interacts with the regulatory protein and renders it active. In one embodiment of the invention the activated regulatory protein interacts with the regulatory element of the expression vector and directs expression of the immunogenic polypeptide.

In a related embodiment of the invention, the regulatory moiety administered is a small molecule. In yet another embodiment of the invention the regulatory moiety administered is a drug. In one aspect of the invention the regulatory moiety is administered orally. In another aspect of the invention the regulatory moiety is administered intravenously.

A further aspect of the invention further comprises administering an adjuvant in addition to the regulatory moiety. In an additional embodiment of the invention the adjuvant is selected from a group consisting of alum, CpG oligonucleotides, guanosine tetraphosphate, guanosine pentaphosphate.

A further embodiment of the invention encompasses a method for producing antibodies which comprises administering: an expression vector containing a DNA sequence encoding at least one immunogenic polypeptide, the expression vector further encoding an expression control sequence, under the control of a regulatory element, capable of directing expression the immunogenic polypeptide; and at least one regulatory moiety, wherein the expression of the immunogenic polypeptide is controlled by a regulatory protein wherein said regulatory protein interacts with the regulatory moiety.

In another related embodiment of the the method, the regulatory protein is endogenously encoded in the animal. In a further embodiment of the invention, the method further comprises administration of the regulatory protein. In another aspect of the the method, the regulatory protein is encoded by a second expression vector. In a related embodiment the method comprises administering the second expression vector.

In a particular aspect of the method, the regulatory moiety interacts with the regulatory protein and renders it active. In a further aspect of the method, the activated regulatory protein interacts with the regulatory element of the expression vector and directs expression of the immunogenic polypeptide.

In one aspect of the method, the regulatory moiety administered is a small molecule. In another aspect of the method, the regulatory moiety administered is a drug. In one embodiment of the method, the regulatory moiety is administered orally. In another embodiment of the invention, the regulatory moiety is administered intravenously. A further embodiment of the method comprises further administering an adjuvant in addition to the regulatory moiety. In a further aspect of the method, the adjuvant is selected from a group consisting of alum, CpG oligonucleotides, guanosine tetraphosphate or guanosine pentaphosphate.

Another embodiment of the invention describes a method for producing an immunogenic polypeptide comprising administering: an expression vector containing a DNA sequence encoding at least one immunogenic polypeptide, the expression vector further encoding an expression control sequence, under the control of a regulatory element, capable of directing expression the immunogenic polypeptide; and at least one regulatory moiety, wherein the expression of the immunogenic polypeptide is controlled by a regulatory protein wherein the regulatory protein interacts with the regulatory moiety.

In a further aspect of the method, the regulatory protein is endogenously encoded in the animal. In a further aspect of the invention the method further comprising administration of the regulatory protein. In a further embodiment of the method comprises administration of a second expression vector wherein the regulatory protein is encoded by the second expression vector.

In one aspect of the invention, the regulatory moiety interacts with the regulatory protein and renders it active. In another aspect of the invention activated the regulatory protein interacts with the regulatory element of the expression vector and directs expression of the immunogenic polypeptide. In one aspect of the method, the regulatory moiety administered is a small molecule. In another aspect of the method, the regulatory moiety administered is a drug. In a further embodiment of the method, the regulatory moiety is administered orally. In a further aspect of the method, the regulatory moiety is administered intravenously. In a further aspect of the method, an adjuvant is administered in addition to the regulatory moiety. In yet another aspect of the method, the adjuvant is selected from a group consisting of alum, CpG oligonucleotides, guanosine tetraphosphate, guanosine pentaphosphate.

In preferred embodiments, the pharmaceutical formulations suitable for administration of the inducible DNA-vaccines developed in this invention are described. Pharmaceutical formulations for different antigens, adjuvants, regulatory moieties and related compounds that may be used with the invention are described. Various modes of delivery of the compounds are also described.

Following long-standing patent law convention, the words “a” and “an” mean “one or more” in this specification, including the claims.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Scheme depicting the inducible vaccine system.

FIG. 2. Construction of inducible vaccine system. DBD: DNA binding domain, which binds to the DBD element; LBD: ligand binding domain, which is functional in the presence of the ligand; AD: activation domain, activates the transcription; ORF: open reading frame.

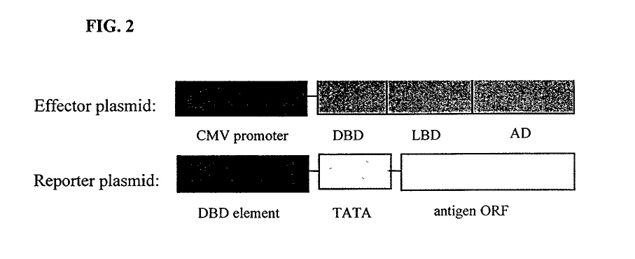

FIG. 3. Anti-hAAT antibody response in vivo.

FIG. 4. Humoral immune response against hAAT in mice.

FIG. 5. Humoral immune response against hAAT in mice where the plasmid DNA was given in 1:1 ratio (1 μg reporter plus 1 μg effector).

FIG. 6. Anti-hAAT antibody production in vivo.

FIG. 7. Anti-hAAT antibody production in mice treated with ecdysone system.

FIG. 8. Inducible plasmids are active for at least 20 weeks after inoculation. Five to 7-week-old female Balb/c mice were given 4 μg reporter (P4U-hAAT) and 200 ng effector (GS1158) of the mifepristone-inducible plasmid DNA divided into four shots. Mifepristone was given i.p. 20 weeks after DNA administration (Day 140, 142, 144). Blood samples were take from the tail vein at various time points and anti-hAAT levels were determined by ELISA. Values are mean +/−SD for three mice per group. The control is with no drug treatment.

FIG. 9. Induction of antigen-specific CTL responses. Balb/c mice (N=3) were immunized with the mifepristone-inducible hAAT coding plasmids and the mifepristone was administered 3 weeks after DNA inoculation. The mice were sacrificed two weeks afterwards and splenocytes were collected. The cells were stimulated with 10 μg/ml denatured hAAT for 5 days in vitro. Lactate dehydrogenase release assays were used to measure cytotoxicity against P815 target cells transfected with pCMVi-hAAT. The values determined had <20% variation. This result demonstrated long-term induction is also true of the cellular response as measured by a cytotoxic T-cell lysis assay of spleen cells from mice induced with drug 3 weeks after inoculation with the inducible hAAT plasmids.

### DESCRIPTION OF ILLUSTRATIVE EMBODIMENTS

A. The Present Invention

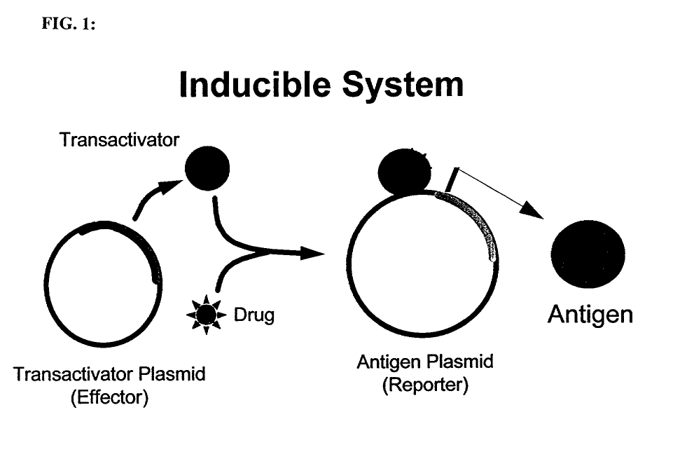

In the present application a vaccine construction has been developed which allows the vaccine to be administered at one time point and the antigen levels to be induced to higher levels at a later time point. The vaccine consists of an antigen open-reading frame under the control of a promoter which is activated by a regulatory protein that is controlled by a small molecule. The regulatory protein could be an endogenous protein or encoded as part of the vaccine. The system in its basic form is depicted in FIG. 1.

This system could be used to boost an immune response to a genetic or conventional vaccine after initial inoculation by simply taking a drug. This system may be particularly useful for vaccination protocols limited by maternal antibodies. For example, pigs could be vaccinated when they are handled young and then given a drug to induce the inducible vaccine at later date. In many cases it is currently not possible to do early vaccination because of the inhibitory effects of maternal antibodies. It may be possible to titrate an immune response over time using varying levels of drug. By using more than one inducible system it may be possible to control the level of two or more antigens at once or in subsequent times.

Inducible expression systems have been developed previously. The inventors used currently available inducible systems and modified them appropriately to be used for vaccination. The unique aspect of this invention is use of these systems for vaccination. The inventors employed the mifepristone (RU486) system (from GeneMedicine), the ecdysone system (Invitrogen, San Diego, Calif.) and the tetracycline system (Life Technologies).

B. DNA-Based Vaccines

A DNA vaccine induces immune responses against an antigenic protein expressed in vivo from an introduced gene. The DNA vaccine is most often in the form of a plasmid DNA expression vector produced in bacteria and then purified and delivered to muscle or skin. DNA vaccines have been demonstrated to show efficacy against numerous viral, bacterial and parasitic diseases in animal models. Almost all studies show induction of very strong and long-lasting humoral and cell-mediated immune responses, and protection against live pathogen challenge (where it could be evaluated). The efficacy of DNA vaccines is attributed, at least in part, to the continuous in vivo synthesis of antigen that leads to efficient antigen presentation. In particular, endogenously-synthesized antigen is presented by class I MHC, leading to induction of CD8⁺ cytotoxic T lymphocytes (CTL). In contrast, most whole killed and subunit vaccines, where antigen is processed solely in the exogenous form, often fail to induce CTL.

DNA-based immunization has several advantages. The antigenic protein is synthesized in vivo giving rise to both humoral and cell-mediated (cytotoxic T lymphocytes) immune responses. However, unlike live attenuated pathogens, which also synthesize protein in vivo, DNA vaccines carry no risk of inadvertent infection. Unlike antigen-based immunization, DNA-based vaccination does not require the use of traditional adjuvants to generate an effective immune response. Furthermore, DNA used in the methods of this invention is inexpensive and easy to manufacture and purify.

DNA-based immunization also allows the host animal to produce foreign antigens within its own tissue thereby resulting in several advantages. One advantage is the efficient presentation of the foreign antigen to the immune system due to the expression of a protein within a self-cell, which could be an antigen-presenting cell. Another advantage is the correct folding, protein modification, and disulfide bonding of a protein expressed in a host cell, especially for viral proteins, which are normally produced in cells of hosts. Recombinant viral proteins synthesized in bacterial or yeast cells may be incorrectly post-translationally modified and are often massed in inclusion bodies, which make the proteins difficult to purify or ineffective if administered in unpurified form.

Another advantage of prolonged synthesis of antigen is the induction of immune responses as soon as the immune system is mature. Animals may be unable to induce sufficient immune responses at a young age. For example, the human fetus and neonate does not produce IgG antibodies and all the IgG is derived from the mother. A human infant produces only about 60% of its adult level of IgG; about 75% of its adult level of IgM and about 20% of its adult level of IgA as late as 12 months after birth. Using the methods of this invention DNA-based immunization can be used as a preferred method for vaccination of young animals.

Species treated by methods of this invention will include a diversity of species of farm animals and humans. Examples of farm animals include, but are not limited to, pigs, cattle, sheep, chickens, turkeys, horses, etc.

C. Adjuvants

Adjuvants for immunization are well known in the art and suitable adjuvants can be combined with the DNA sequences described herein by a person skilled in the art to form a pharmaceutical composition. Oil adjuvants are least desirable for the methods of this invention because they create undesirable side-effects such as visceral adhesions (which can restrict growth) and melanized granuloma formations (which can lower the grade of the animals at market) and because they cannot form a homogeneous mixture with DNA preparations. DNA-based immunization does not require oil adjuvants and thus avoids these undesirable effects. Adjuvants used in immunization with DNA expression plasmids of this invention may include alum or a DNA molecule having unmethylated CpG dinucleotides therein (also referred to as CpG adjuvant). Oligonucleotides having unmethylated CpG dinucleotides have been shown to activate the immune system (Krieg, et al., 1995). CpG motifs may be inserted into a plasmid DNA vaccine vector, and replicated in bacteria thereby allowing the CpG motifs to retain their unmethylated form. As such, administration of a CpG adjuvant cloned into plasmid vectors would be simultaneous with the administration of a plasmid DNA vaccine. Alternatively, a CpG adjuvant in the form of free oligonucleotides may be administered before, during or after the administration of a plasmid DNA vaccine. Oligonucleotides having CpG motifs may be optionally modified at their phosphodiester linkages for stability purposes. Such modifications are well known by those of skill in the art. For example, phosphodiester bonds in an oligonucleotide may be replaced by phosphorothioate linkages.

Two bacterial nucleotides, guanosine tetraphosphate (ppGpp) and guanosine pentaphosphate (pppGpp), can also stimulate the vertebrate immune system to mount a rapid immune response against a specific antigen. Co-administration of an antigen, alum and ppGpp or a mixture of ppGpp and pppGpp dramatically decreases the time required to elicit an immune response against the antigen. Guanosine tetra- and pentaphosphate or simple derivatives may have broad use as adjuvants, as stimulants of the innate immune response, and as modifiers of the character of an immune response and have the potential to be used clinically as adjuvants in humans and animals for immunotherapy, and for prophylactic and therapeutic vaccination against infectious diseases (see copending U.S. Serial No. 60/129,665 by Irene T. Rombel filed Apr. 15, 1999).

This invention is not limited to the adjuvants described herein and any other adjuvant or hapten may be used in combination with the DNA-based vaccines of this invention.

### D. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Types of Antigen Encoded by DNA Vaccines

DNA which is introduced to animals species will encode foreign polypeptides (e.g., those derived from viral, bacterial or parasitic pathogens) or peptides derived from tumors including tumor markers. Polypeptides of this invention refer to complete proteins or fragments thereof, including peptides which are epitopes (e.g., a CTL epitope) associated with an infectious virus, bacterium or parasite; or a tumor marker. An “antigenic polypeptide” or “antigenic protein” is any polypeptide/protein that can, under appropriate conditions, induce an immune response. Minor modifications of the primary amino acid sequences of a viral, bacterial, pathogenic or tumor polypeptide may also result in a polypeptide which have substantially equivalent antigenic activity as compared to the unmodified counterpart polypeptide. Such modifications may be deliberate, as by site-directed mutagenesis, or other techniques known in the art, or may be spontaneous. All of the polypeptides produced by these modifications are included herein as long as antigenicity still exists.

DNA sequences encoding a complete or large parts of an antigenic protein are preferred where humoral immunity is desired rather than DNA sequences encoding smaller parts, such as only CTL epitopes, as are preferred where cell-mediated immunity is desired and humoral immunity may be deleterious. In preferred embodiments, the DNA sequences encoding immunogenic polypeptides of viral pathogens may be selected from the group consisting of viral surface antigens such as but not limited to glycoprotein (G) or nucleoprotein (N) of any viral species.

In other preferred embodiments, the DNA sequences encoding immunogenic polypeptides of bacterial pathogens may be selected.

In yet another preferred embodiment, the DNA sequences encoding immunogenic polypeptides of a parasitic pathogen may be selected.

In other preferred embodiments, the DNA sequences encoding polypeptides of a tumor may be selected from the cancer/tumor surface antigens such as but not limited to the oncogenic proteins of the MAGE, PAGE and GAGE families.

One of skill in the art will recognize that there are many vaccines that can be used with regard to this invention in addition to those described above.

### Example 2

### Recombinant Vectors, Host Cells and Expression

The term “expression vector or construct” means any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product.

Particularly useful vectors are contemplated to be those vectors in which the coding portion of the DNA-vaccine, whether encoding a full length protein, polypeptide or smaller peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control” or “under transcriptional control” means that the promoter is in the correct location and orientation in relation to the nucleic acid to control RNA polymerase initiation and expression of the gene.

The promoter may be in the form of the promoter that is naturally associated with an gene, as may be obtained by isolating the 5′ non-coding sequences located upstream of the coding segment or exon, for example, using recombinant cloning and/or PCR® technology, in connection with the compositions disclosed herein (PCR® technology is disclosed in U.S. Pat. No. 4,683,202 and U.S. Pat. No. 4,682,195, each incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a gene in its natural environment. Such promoters may include promoters normally associated with other genes, and/or promoters isolated from any other bacterial, viral, eukaryotic, or mammalian cell, and/or promoters made by the hand of man that are not “naturally occurring,” i.e., containing difference elements from different promoters, or mutations that increase, decrease, or alter expression.

Naturally, it will be important to employ a promoter that effectively directs the expression of the DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of the introduced DNA segment, such as is advantageous in the large-scale production of recombinant proteins or peptides.

At least one module in a promoter generally functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of the instant nucleic acids. The use of other viral or mammalian cellular or bacterial phage promoters which are well-known in the art to achieve expression are contemplated as well, provided that the levels of expression are sufficient for a given purpose. Tables 1 and 2 below list several elements/promoters which may be employed, in the context of the present invention, to regulate the expression of a gene. This list is not intended to be exhaustive of all the possible elements involved in the promotion of expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
Promoter and Enhancer Elements

| Promoter/Enhancer | References |
|---|---|
| Immunoglobulin Heavy | Banerji et al., 1983; Gilles et al., 1983; |
| Chain | Grosschedl and Baltimore, 1985; Atchinson |
| | and Perry, 1986, 1987; Imler et al., 1987; |
| | Weinberger et al., 1984; Kiledjian et al., |
| | 1988; Porton et al.; 1990 |
| Immunoglobulin Light | Queen and Baltimore, 1983; Picard and |
| Chain | Schaffner, 1984 |
| T-Cell Receptor | Luria et al., 1987; Winoto and Baltimore, |
| | 1989; Redondo et al.; 1990 |
| HLA DQ a and DQ β | Sullivan and Peterlin, 1987 |
| β-Interferon | Goodbourn et al., 1986; Fujita et al., 1987; |
| | Goodbourn and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase | Jaynes et al., 1988; Horlick and Benfield, |
| | 1989; Johnson et al., 1989 |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, |
| | 1990 |
| α-Fetoprotein | Godbout et al., 1988; Campere and |
| | Tilghman, 1989 |
| t-Globin | Bodine and Ley, 1987; Perez-Stable and |
| | Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α-_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al, 1986 |
| Human Serum Amyloid | Edbrooke et al., 1989 |
| A (SAA) | |
| Troponin I (TN I) | Yutzey et al., 1989 |
| Platelet-Derived Growth | Pech et al., 1989 |
| Factor | |
| Duchenne Muscular | Klamut et al., 1990 |
| Dystrophy | |
| SV40 | Banerji et al., 1981; Moreau et al., 1981; |
| | Sleigh and Lockeft, 1985; Firak and |
| | Subramanian, 1986; Herr and Clarke, 1986; |
| | Imbra and Karin, 1986; Kadesch and Berg, |
| | 1986; Wang and Calame, 1986; Ondek et al., |
| | 1987; Kuhl et al., 1987; Schaffner et al., |
| | 1988 |
| Polyoma | Swartzendruber and Lehman, 1975; Vasseur |
| | et al., 1980; Katinka et al., 1980, 1981; |
| | Tyndell et al., 1981; Dandolo et al., 1983; de |
| | Villiers et al., 1984; Hen et al., 1986; Satake |
| | et al., 1988; Campbell and Villarreal, 1988 |
| Retroviruses | Kriegler and Botchan, 1982, 1983; Levinson |
| | et al., 1982; Kriegler et al., 1983, 1984a, b, |
| | 1988; Bosze et al., 1986; Miksicek et al., |
| | 1986; Celander and Haseltine, 1987; Thiesen |
| | et al., 1988; Celander et al., 1988; Chol et |
| | al, 1988; Reisman and Rotter, 1989 |
| Papilloma Virus | Campo et al., 1983; Lusky et al., 1983; |
| | Spandidos and Wilkie, 1983; Spalholz et al., |
| | 1985; Lusky and Botchan, 1986; Cripe et al., |
| | 1987; Gloss et al., 1987; Hirochika et al., |
| | 1987; Stephens and Hentschel, 1987; Glue et |
| | al., 1988 |
| Hepatitis B Virus | Bulla and Siddiqui, 1986; Jameel and |
| | Siddiqui, 1986; Shaul and Ben-Levy, 1987; |
| | Spandau and Lee, 1988; Vannice and |
| | Levinson, 1988 |
| Human Immunodeficiency | Muesing et al., 1987; Hauber and Cullan, |
| Virus | 1988; Jakobovits et al., 1988; Feng and |
| | Holland, 1988; Takebe et al., 1988; Rosen |
| | et al., 1988; Berkhout et al., 1989; Laspia |
| | et al., 1989; Sharp and Marciniak, 1989; |
| | Braddock et al., 1989 |
| Cytomegalovirus | Weber et al., 1984; Boshart et al., 1985; |
| | Foecking and Hofstetter, 1986 |
| Gibbon Ape Leukemia | Holbrook et al., 1987; Quinn et al., 1989 |
| Virus | |

TABLE 2
Inducible Elements

| Element | Inducer | References |
|---|---|---|
| MT II | Phorbol Ester (TFA) | Palmiter et al., 1982; Haslinger and |
| | Heavy metals | Karin, 1985; Searle et al., 1985; |
| | | Stuart et al., 1985; Imagawa et al., |
| | | 1987, Karin et al., 1987; Angel et |
| | | al., 1987b; McNeall et al., 1989 |
| MMTV (mouse | Glucocorticoids | Huang et al., 1981; Lee et al., |
| mammary | | 1981; Majors and Varmus, 1983; |
| tumor virus) | | Chandler et al., 1983; Lee et al., |
| | | 1984; Ponta et al., 1985; Sakai et |
| | | al., 1988 |
| β-Interferon | poly(rI)x | Tavernier et al., 1983 |
| | poly(rc) | |
| Adenovirus | Ela | Imperiale and Nevins, 1984 |
| 5 E2 | | |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX | Interferon, | |
| Gene | Newcastle Disease | |
| | Virus | |
| GRP78 Gene | A23187 | Resendez et al., 1988 |
| α-2- | IL-6 | Kunz et al., 1989 |
| Macroglobulin | | |
| Vimentin | Serum | Riffling et al., 1989 |
| MHC Class I | Interferon | Blanar et al., 1989 |
| Gene H-2κb | | |
| HSP70 | Ela, SV40 Large | Taylor et al., 1989; Taylor and |
| | T Antigen | Kingston, 1990a, b |
| Proliferin | Phorbol Ester-TPA | Mordacq and Linzer, 1989 |
| Tumor | FMA | Hensel et al., 1989 |
| Necrosis Factor | | |
| Thyroid Stim- | Thyroid Hormone | Chatterjee et al., 1989 |
| ulating Hor- | | |
| mone a Gene | | |

Turning to the expression of the proteins, once a suitable clone or clones have been obtained, whether they be cDNA based or genomic, one may proceed to prepare an expression system. The engineering of DNA segment(s) for expression in a prokaryotic or eukaryotic system may be performed by techniques generally known to those of skill in recombinant expression. It is believed that virtually any expression system may be employed in the expression of the proteins.

Both cDNA and genomic sequences are suitable for eukaryotic expression, as the host cell will generally process the genomic transcripts to yield functional mRNA for translation into protein. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude or more larger than the cDNA gene. However, it is contemplated that a genomic version of a particular gene may be employed where desired.

In expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

It is proposed that proteins, polypeptides or peptides may be co-expressed with other selected proteins, wherein the proteins may be co-expressed in the same cell or an gene(s) may be provided to a cell that already has another selected protein. Co-expression may be achieved by co-transfecting the cell with two distinct recombinant vectors, each bearing a copy of either of the respective DNA. Alternatively, a single recombinant vector may be constructed to include the coding regions for both of the proteins, which could then be expressed in cells transfected with the single vector. In either event, the term “co-expression” herein refers to the expression of both the gene(s) and the other selected protein in the same recombinant cell.

As used herein, the terms “engineered” and “recombinant” cells or host cells are intended to refer to a cell into which an exogenous DNA segment or gene, such as a cDNA or gene encoding an protein has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant protein, polypeptide or peptide, whether mutant or wild-type, in accordance with the present invention one would prepare an expression vector that comprises a wild-type, or mutant protein-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded recombinant protein. This is the meaning of “recombinant expression” in this context.

Many standard techniques are available to construct expression vectors containing the appropriate nucleic acids and transcriptional/translational control sequences in order to achieve protein, polypeptide or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more protein, polypeptide or peptide coding sequences.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the gene sequence(s), provided such control sequences are compatible with the host cell systems.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the Bg1I site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1, E3, or E4) will result in a recombinant virus that is viable and capable of expressing proteins, polypeptides or peptides in infected hosts.

Specific initiation signals may also be required for efficient translation of protein, polypeptide or peptide coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements and transcription terminators.

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

For long-term, high-yield production of a recombinant protein, polypeptide or peptide, stable expression is preferred. For example, cell lines that stably express constructs encoding an protein, polypeptide or peptide by the methods disclosed herein may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including, but not limited to, the herpes simplex virus thymidine kinase (tk), hypoxanthine-guanine phosphoribosyltransferase (hgprt) and adenine phosphoribosyltransferase (aprt) genes, in tk⁻, hgprt⁻ or aprt⁻ cells, respectively. Also, antimetabolite resistance can be used as the basis of selection for dihydrofolate reductase (dhfr), that confers resistance to methotrexate; gpt, that confers resistance to mycophenolic acid; neomycin (neo), that confers resistance to the aminoglycoside G-418; and hygromycin (hygro), that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

### Example 3

### Expression of Antigenic Regions for DNA-Based Vaccination

The general approach to the aspects of the present invention concerning preventative therapeutics is to provide a cell with a gene construct encoding a specific or desired antigen which is either a protein, polypeptide or peptide. While it is conceivable that the gene construct and/or protein may be delivered directly, a preferred embodiment involves providing a nucleic acid encoding a specific or desired protein, polypeptide or peptide to the cell. Following this provision, the proteinaceous composition is synthesized by the transcriptional and translational machinery of the cell, as well as any that may be provided by the expression construct.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

### Example 4

### Methods for Delivering DNA-Based Vaccination

In order to effect expression of a gene construct, the DNA-based vaccine expression construct must be delivered into a cell. As used herein, “gene” may refer to an encoded antigen expressed by a DNA-based vaccine construct. The DNA-based vaccine expression construct may consist only of naked recombinant DNA or plasmids. Transfer of the construct may be performed by any of the methods mentioned which physically or chemically permeabilize the cell membrane. Some of these techniques may be successfully adapted for in vivo or ex vivo use, as discussed below.

The nucleic acid encoding the antigen may be stably integrated into the genome of the cell, or may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

The production of immune response in the preferred embodiment of the invention include administration of a regulatory moiety or an activating agent that activates another regulatory protein (encoded naturally by the host or transgenically encoded), which is capable of inducing the expression of the antigen. The regulatory moiety could be but not limited to a drug or a small molecule. The production and titre of antibodies produced may be monitored by sampling blood of the vaccinated animal or human at various points following administration of the DNA-based vaccine. One or more additional booster doses of regulatory moiety may also be given. The animal's ability to mount an immune response to an antigen may be measured by administration of one or more doses of the antigen, collecting the animal's serum, and tittering the serum for antibodies to the antigen, using techniques known to those of ordinary skill in the art. The process of boosting and tittering is repeated until a suitable antibody titer is achieved, i.e. the animal mounts an effective immune response to a particular antigen.

A. Particle Bombardment

As described above, a preferred method of introducing a DNA-based vaccine expression construct into cells is through particle bombardment. This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads. However, the inventors have had particular success using the following general protocol described above.

B. Injection

DNA-based vaccines may be introduced by needle injection in a saline solution. Methods of injection of vaccines are well known to those of ordinary skill in the art. The amount of DNA-based vaccine used may vary upon the nature of the antigen as well as the animal used. A variety of routes can be used to administer the antigen (subcutaneous, intramuscular, intradermal, intravenous and intraperitoneal).

C. Liposome-Mediated Transfection

The DNA-based vaccine expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

The liposome may be complexed with a hemagglutinating virus (HVJ). This has been shown to facilitate fusion with the cell membrane and promote cell entry of liposome-encapsulated DNA (Kaneda et al., 1989). The liposome may be complexed or employed in conjunction with nuclear non-histone chromosomal proteins (HMG-1) (Kato et al., 1991). The liposome may be complexed or employed in conjunction with both HVJ and HMG-1. The delivery vehicle may comprise a ligand and a liposome.

D. Electroporation

The DNA-based vaccine expression construct may be introduced into the cell via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

E. Calcium Phosphate or DEAE-Dextran

The DNA-based vaccine expression construct may be introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

The DNA-based vaccine expression construct may be delivered into the cell using DEAE-dextran followed by polyethylene glycol. In this manner, reporter plasmids were introduced into mouse myeloma and erythroleukemia cells (Gopal, 1985).

F. Direct Microinjection or Sonication Loading

The introduction of the DNA-based vaccine expression construct may be introduced into a cell by direct microinjection or sonication loading. Direct microinjection has been used to introduce nucleic acid constructs into Xenopus oocytes (Harland and Weintraub, 1985), and LTK⁻ fibroblasts have been transfected with the thymidine kinase gene by sonication loading (Fechheimer et al., 1987).

G. Adenoviral Assisted Transfection

The DNA-based vaccine expression construct may be introduced into the cell using adenovirus assisted transfection. Increased transfection efficiencies have been reported in cell systems using adenovirus coupled systems (Kelleher and Vos, 1994; Cotten et al., 1992; Curiel, 1994).

H. Receptor Mediated Transfection

DNA-based vaccine expression constructs that may be employed to deliver nucleic acid construct to target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of another mammalian cell type is described by Wu and Wu (1993; incorporated herein by reference).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. The ligand may be chosen to correspond to a receptor specifically expressed on the EOE target cell population.

The DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

The DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the tissue-specific DNA-based vaccine constructs of the present invention can be specifically delivered into the target cells in a similar manner.

I. DNA-Based Vaccine Delivery Using Viral Vectors

The ability of certain viruses to infect cells or enter cells via receptor-mediated endocytosis, and to integrate into host cell genome and express viral genes stably and efficiently have made them attractive candidates for the transfer of foreign genes into mammalian cells. DNA-based vaccine vectors of the present invention may be viral vectors that encode antigens.

1. DNA-Based Vaccine Delivery Using Adenoviral Vectors

A particular method for delivery of the DNA-based vaccine expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein.

The DNA-based vaccine expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

2. DNA-Based Vaccine Delivery Using AAV Vectors

Adeno-associated virus (AAV) is an attractive vector system for use in the DNA-based vaccines of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

3. DNA-Based Vaccine Delivery Using Retroviral Vectors

Retroviruses have promise as antigen delivery vectors in DNA-based vaccines due to their ability to integrate their genes into the host genome, transferring a large amount of foreign genetic material, infecting a broad spectrum of species and cell types and of being packaged in special cell-lines (Miller, 1992).

In order to construct a DNA-based vaccine retroviral vector, a nucleic acid encoding a antigen of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Gene delivery using second generation retroviral vectors has been reported. Kasahara et al (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

4. DNA-Based Vaccine Delivery Using Other Viral Vectors

Other viral vectors may be employed as DNA-based vaccine expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. DNA-Based Vaccine Delivery Using Modified Viruses

The nucleic acids to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989). Thus, it is contemplated that antibodies, specific binding ligands and/or other targeting moieties may be used to specifically transfect non-APC types.

### Example 5

### Pharmaceutical Compositions

The present invention also includes pharmaceutical products for all of the uses contemplated in the methods described herein. For example, a pharmaceutical product comprising pure plasmid DNA vector or formulations thereof, operatively coding for an antigen i.e. an immunogenic protein, polypeptide or peptide, may be prepared in physiologically acceptable administrable form (e.g., saline). Other pharmaceutical products required for the synthesis of a vaccine include adjuvants, haptens, regulatory moieties, small molecules, drugs, carriers, and formulations thereof. The pharmaceutical product may be placed in a container, with a notice associated with the container in the form prescribed by a governmental agency regulating the manufacture, use or sale of pharmaceuticals, which notice is reflective of approval by the agency of the form of the DNA for veterinary administration. Such notice, for example, may be labeling approved by the United States Department of Agriculture (USDA).

A. Pharmaceutically Acceptable Carriers

Aqueous compositions comprise an effective amount of the specific or desired gene construct, protein, polypeptide, peptide, or adjuvant, hapten or such like dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, subcutaneous, intralesional, or even intraperitoneal routes. The preparation of an aqueous composition that contains an specific or desired agent as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An specific or desired gene construct, protein, polypeptide and/or peptide can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodernoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

B. Liposomes and Nanocapsules

In certain embodiments, the use of liposomes and/or nanoparticles is contemplated for the introduction of specific or desired protein, polypeptides, peptides or agents, or gene-based vaccine vectors into host cells. The formation and use of liposomes is generally known to those of skill in the art, and is also described below.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

C. Pharmaceutical Formulations of the Regulatory Moiety

The regulatory moiety or activating agent described in the invention which can induce antigen levels to higher levels at a later time point can be a small molecule or drug which activates a regulatory protein which could be an endogenous protein or encoded as part of the vaccine. In a preferred embodiment this regulatory moiety will be an oral drug and the pharmaceutical formulations for this embodiment are described herein.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

### Example 6

Humoral Immune Response Against hAAT in Mice

Specific-pathogen-free (5-7 weeks old), female balb/c mice were purchased from Jackson Labs (Bar Harbor, Me.). A biolistic gene gun (Rumsey-Loomis, Ithaca, N.Y.) was used to deliver plasmid DNA into the skin of mouse ears (Williams et al., 1991; Johnston and Tang 1993). Briefly, DNA was precipitated onto gold micro-projectiles (3±1 μm diameter, Metz Metallurgical Corp., South Plainfield, N.J.) at 4 μg DNA/mg gold. Each mouse received four shots of gold-DNA and each shots contains 1 μg reporter (P4U-AAT) and 50 ng effector (GS-1158) plasmid DNA. The drug (RU486, Sigma) was given intraperitoneally on days 1, 3, 5, and also on day 14, 16, in a dose of 500 μg/kg/day in 100 μl sesame oil, after DNA injection. CMVi-AAT plasmid was given as an antigen boost on Week 11 after the primary DNA inoculation. The blood samples of the mice was collected from the tail vein at the time point as indicated. The anti-hAAT antibodies in the serum was measured using enzyme-linked immuno enzyme-linked immunosorbent assay (ELISA, Tang et al. 1992). Briefly, ninety six-well plats were coated with 1 μg hAAT per well in 50 μl phosphate buffered saline (PBS) (for overnight at 4° C. and 100 μl blocking buffer (3% bovine serum albumin (3SA) in PBS with 0.05% tween 20) was added and incubated for 2 hrs at room temperature. After 3 times washing with PBS plus 0.05% tween 20, the serum samples (diluted 1:200 in blocking buffer) were added into the well and incubated overnight at 4° C., then further incubated with horseradish peroxidase-conjugated goat anti-mouse immunoglobulin G and M (Jackson ImmunResearch, West Grove, Pa.) for 1 h at room temperature. The plate was washed and developed with TMB buffer solution (Calbiochem). The optical density was read at 450 nm and concentration of the antibodies was determined according to the standard curve.

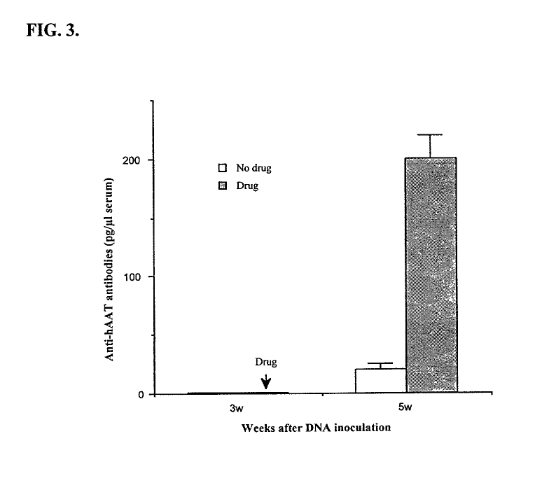

FIG. 3 depicts the humoral immune response against hAAT in mice. The value (N=3) showed has been subtracted from the concurrent mock DNA inoculated control mice (N=2). In RU486 treated group, the anti-hAAT antibody reached peak levels at the third week. Although the antibody levels falls afterwards, second exposure to the antigen (CMVi-AAT on 11 week) ensued an secondary immune response, indicating the immune system remains in a memory state, not tolerance.

### Example 7

### Anti-hAAT Antibody Response in vivo

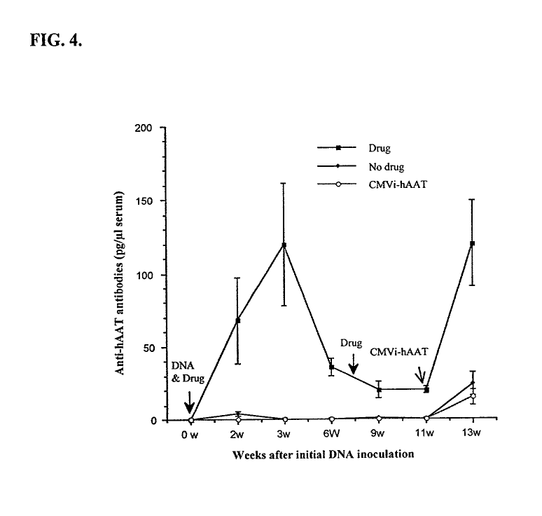

Methods described in Example 6 were used. The ligand (RU486) was given on week 3 (day 21, 23, 25) after DNA injection (4 shoots each with 1 μg reporter and 50 ng effector plasmid DNA each mouse. FIG. 4 shows that the immune response is inducible by simply giving the ligand three weeks after the primary injection of the DNA. The value (N=3) showed has been subtracted from the concurrent mock DNA inoculated control mice (N=2).

### Example 8

### Anti-hAAT Antibody Response in vivo where the Plasmid DNA was Administered in a 1:1 Ratio

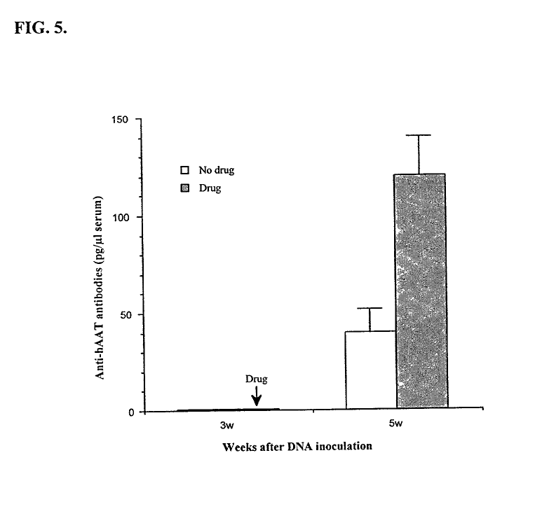

Methods described in Example 6 were used. FIG. 5 depicts the response of the anti-hAAT antibody when the plasmid DNA was administered in a 1:1 ratio (1 μg reporter plus 1 μg effector for each shot). In this condition, high background antibody levels was seen in the non-drug treated group but the immune response in the drug treated group was still significantly induced.

### Example 9

### Anti-hAAT Antibody Production in vivo

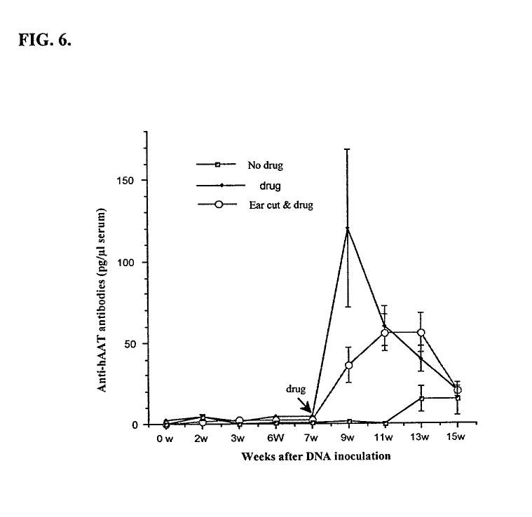

Methods described in Example 6 were used. FIG. 6 shows the production of the anti-hAAT antibody in vivo. The mice were given RU486 intraperitoneally in week 7 (Day 50, 52, 54) after the DNA injection in the drug and the Van Gogh (removed the site of the DNA inoculation, ear) groups. The ear cut was performed before administering the drug. The value shown is the mean with standard deviation for experiments on three mice. The immune response was inducible 7 weeks after the DNA injection by giving the ligand in mice and the original vaccine site was not required for induction.

### Example 10

### Anti-hAAT Antibody Production in Mice Treated with Ecdysone System

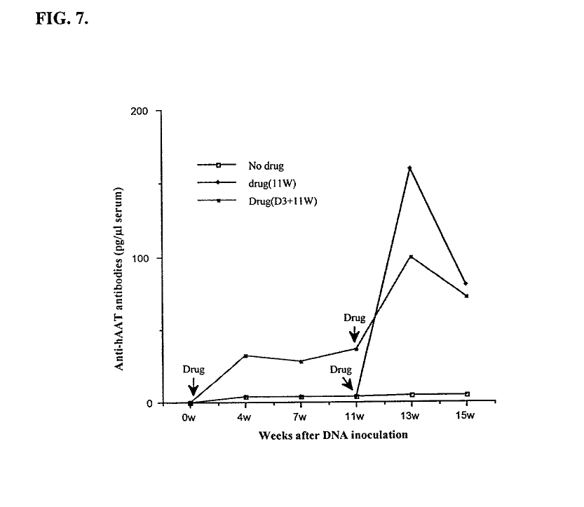

FIG. 7 depicts the effect of ecdysone system treatment. The ligand (muristerone, 3 mg) was given ip on 11 weeks after inoculating the ecdysone regulatory plasmid system. The mice received four shots each containing 1 μg reporter plus 1 μg effector plasmid DNA, The value showed has been subtracted from the value of concurrent mock DNA treated control mice (N=2). For detailed experimental method see Example 6.

### Example 11

### Anti-hAAT Antibody Production in Mice by Mifepristone-Inducible Plasmid DNA

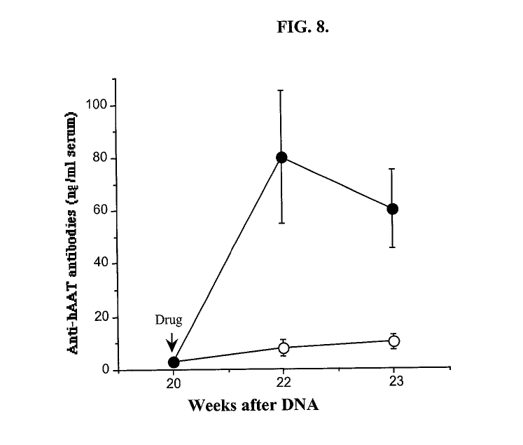

The study shown in FIG. 8, demonstrates that the mifepristone-inducible plasmids are active for at least 20 weeks after inoculation. Here the mice were inoculated with the induction system and drug administered at week 20 after the plasmids were delivered. As evident in FIG. 4, the drug induced a significant antibody response relative to the non-drug control.

### Example 12

### Induction of Antigen-Specific CTL Responses

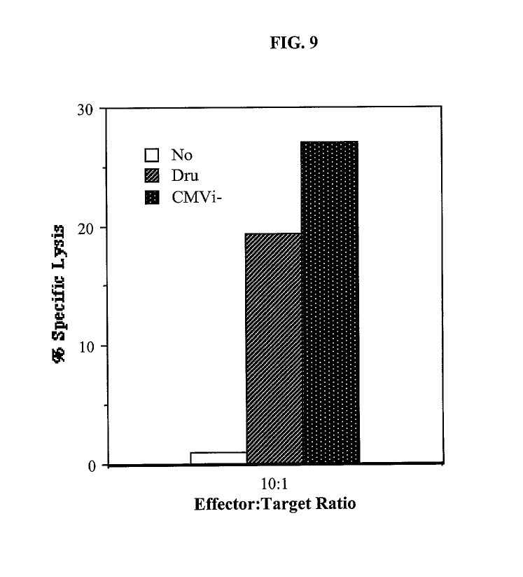

The induction of antigen-specific CTL responses is shown in FIG. 9 in this study where Balb/c mice (N=3) were immunized with the mifepristone-inducible hAAT coding plasmids and the mifepristone was administered 3 weeks after DNA inoculation. The mice were sacrificed two weeks afterwards and splenocytes were collected. The cells were stimulated with 10 μg/ml denatured hAAT for 5 days in vitro. Lactate dehydrogenase release assays were used to measure cytotoxicity against P815 target cells transfected with pCMVi-hAAT. The values determined had <20% variation. This result demonstrated long-term induction is also true of the cellular response as measured by a cytotoxic T-cell lysis assay (described below) of spleen cells from mice induced with drug 3 weeks after inoculation with the inducible hAAT plasmids.

Method for CTL Assay

To measure cytotoxicity, spleen cells were cultured for five days in the presence of 10 μg/ml of heat denatured (95° C., 10 min.) hAAT protein. Viable cells were harvested from these cultures by centrifugation over Ficoll-Paque (Pharmacia, Uppsala, Sweden) and examined for their cytotoxic activity. P815 cell line that had been transfected with pCMVi-hAAT. Specific cytotoxicity was examined by measuring LDH release after a 4-hour incubation of splenic cells and targets (5×10³ cells/well) (Promega).

### Example 13

### Optimization of an Inducible Vaccine

Crucial to the effectiveness of an inducible vaccine can be the titration of the amount of regulatory plasmid against the amount of reporter plasmid. Table 3 depicts the titration/optimization performed by the inventors which demonstrates that all three systems can work in vivo using a luciferase (luc) reporter system. Experiments by the inventors shows that a vector system should produce at least 5×10⁶ lumens to be readily capable of inducing an immune response. The production of less than 1×10⁵ lumens does not generate an immune response even if multiple boosts are tried. Given this window of induction the amount of each plasmid in each system was varied to obtain combinations that fell in this window. The inventors also tested constructs with and without introns and with variations of the TATA box position. The inventors found combinations with each inducible system that gave induction that spanned the immunological window.

With this knowledge in hand the inventors tested an inducible vaccine. Mice were injected with the inducible plasmids or control plasmids. A negative control was a plasmid not encoding a protein, and a positive control was a plasmid where the CMV promoter was controlling the human alpha1-anti-trypsin gene (hAAT). The inducible plasmids also encode hAAT. In one group of mice the inducer drug was given right after gene immunization. In other mice the inducer drug was given at various time intervals after the initial inoculation. Several sets of experiments were performed and are presented in FIGS. 3-7.

TABLE 3
Luciferase expression in mouse ear after inoculation
of the drug-indictable plasmid systems

| | DNA amounts | Luc activity | Luc activity | Fold of |
|---|---|---|---|---|
| Vectors | (Repoter + Effector) | (No Drug) | (Drug) | Induction |
| RU486 | 1 μg + 0 | 6.4 × 10⁴ | 6.1 × 10⁴ | 0 |
| System | 1 μg + 10 ng | 9.7 × 10⁴ | 7.3 × 10⁵ | 7.5 |
| | 1 μg + 50 ng | 8.5 × 10⁴ | 9.6 × 10⁶ | 113 |
| | 1 μg + 100 ng | 4.4 × 10⁵ | 8.7 × 10⁶ | 20 |
| | 1 μg + 1 μg | 1.3 × 10⁶ | 1.8 × 10⁷ | 14 |
| Tet-Off | 1 μg + 0 | 1.2 × 10⁵ | 1.2 × 10⁵ | 0 |
| System | 1 μg + 50 ng | 7.5 × 10⁵ | 4.4 × 10⁴ | 17 |
| | 1 μg + 100 ng | 2.3 × 10⁶ | 7.5 × 10⁴ | 30 |
| | 1 μg + 1 μg | 2.3 × 10⁶ | 7.6 × 10⁴ | 30 |
| Tet-On | 1 μg + 50 ng | 5.9 × 10³ | 4.3 × 10⁴ | 7 |
| System | 1 μg + 100 ng | 8.4 × 10⁴ | 1.7 × 10⁶ | 20 |
| | 1 μg + 1 μg | 6.2 × 10⁴ | 4.5 × 10⁵ | 7.2 |
| Ecdysone | 1 μg + 0 | 8.7 × 10⁴ | NA | — |
| System | 1 μg + 10 ng | 1.3 × 10⁵ | NA | — |
| | 1 μg + 50 ng | 2.1 × 10⁵ | NA | — |
| | 1 μg + 100 ng | 2.2 × 10⁵ | 4.5 × 10⁶ | 20 |
| | 1 μg + 1 μg | 1.3 × 10⁵ | 4.5 × 10⁶ | 33 |

The mouse (Balb/c) ears were cotransfected with 1 μg reporter plasmid DNA and various amount of effector plasmid DNA as indicated by gene-gun method. The RU486 (500 μg/kg), doxycycline (1 mg per mouse), and Muristerone (3 mg per mouse) was given ip in 100 μl sesame oil to the mice 1 hr after DNA injection and Luciferase activity of each ear was measured 24 hrs later using a standard method (Promega). The value of the luciferase activity is the means of four mouse ears in an arbitrary unite. The standard error in all group is within 10-30%.

The inducible vaccine described herein was functional and an immune response was inducible up to at least 11 wk after the initial injection. Thus, one can engineer a system such that even after multiple injections no immune response is induced in the absence of the inducer drug. The Van Gogh experiment (FIG. 6) demonstrates that a local vaccine site is not required for induction. However, a local vaccine site increases the level of immune response.

Even though the system could not be induced more than once in these examples, the immune response can be boosted with another introduction of plasmid. The lack of second induction does not, therefore, reflect production of tolerance.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Acsadi et al., New Biologist, 3:71-81, 1991.

Angel et al., “12-0-tetradecanoyl-phorbol-13-acetate Induction of the Human Collagenase Gene is Mediated by an Inducible Enhancer Element Located in the 5′ Flanking Region,” Mol. Cell. Biol., 7:2256, 1987a.

Angel et al., “Phorbol Ester-Inducible Genes Contain a Common cis Element Recognized by a TPA-Modulated Trans-acting Factor,” Cell, 49:729, 1987b.

Atchison and Perry, “Tandem Kappa Immunoglobulin Promoters are Equally Active in the Presence of the Kappa Enhancer: Implications for Model of Enhancer Function,” Cell, 46:253, 1986.

Atchison and Perry, “The Role of the Kappa Enhancer and its Binding Factor NF-kappa B in the Developmental Regulation of Kappa Gene Transcription,” Cell, 48:121, 1987.

Banerji et al., “A Lymphocyte-Specific Cellular Enhancer is Located Downstream of the Joining Region in Immunoglobulin Heavy-Chain Genes,” Cell, 35:729, 1983.

Banerji et al., “Expression of a Beta-Globin Gene is Enhanced by Remote SV40 DNA Sequences,” Cell, 27:299, 1981.

Berkhout et al., “Tat Trans-activates the Human Immunodeficiency Virus Through a Nascent RNA Target,” Cell, 59:273, 1989.

Blanar et al., “A Gamma-Interferon-Induced Factor That Binds the Interferon Response Sequence of the MHC Class I Gene, H-2 Kb,” EMBO J, 8:1139, 1989.

Bodine and Ley, “An Enhancer Element Lies 3′ to the Human A Gamma Globin Gene,” EMBO J, 6:2997, 1987.

Boshart et al., “A Very Strong Enhancer is Located Upstream of an Immediate Early Gene of Human Cytomegalovirus,” Cell, 41:521, 1985.

Bosze et al., “A Transcriptional Enhancer with Specificity for Erythroid Cells is Located in the Long Terminal Repeat of the Friend Murine Leukemia Virus,” EMBO J, 5:1615, 1986.

Braddock et al., “HIV-I Tat Activates Presynthesized RNA I nthe Nucleus,” Cell, 58:269, 1989.

Bulla and Siddiqui, “The Hepatitis B Virus Enhancer Modulates Transcription of the Hepatitis B Virus Surface-Antigen Gene From an Internal Location,” J. Virol., 62:1437, 1986.

Campbell and Villarreal, “Functional Analysis of the Individual Enhancer Core Sequences of Polyoma Virus: Cell-Specific Uncoupling of DNA Replication From Transcription,” Mol. Cell. Biol., 8:1993, 1988.

Campere and Tilghman, “Postnatal Repression of the α-fetoprotein Gene is Enhancer Independent,” Genes and Dev., 3:537, 1989.

Campo et al., “Transcriptional Control Signals in the Genome of Bovine Papilloma Virus Type 1,” Nature, 303:77, 1983.

Caplan et al., Nature Med., 1:39-46, 1995.

Celander and Haseltine, “Glucocorticoid Regulation of Murine Leukemia Virus Transcription Elements is Specified by Determinants Within the Viral Enhancer Region,” J. Virology, 61:269, 1987.

Celander et al., “Regulatory Elements Within the Murine Leukemia Virus Enhancer Regions Mediate Glucocorticoid Responsiveness,” J. Virology, 62:1314, 1988.

Chandler et al., “DNA Sequences Bound Specifically by Glucocorticoid Receptor in vitro Render a Heterlogous Promoter Hormone Responsive in vivo,” Cell, 33:489, 1983.

Chang et al., “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee et al., “Negative Regulation of the Thyroid-Stimulating Hormone Alpha Gene by Thyroid Hormone: Receptor Interaction Adjacent to the TATA Box,” Proc.

Natl. Acad. Sci. U.S.A., 86:9114, 1989.

Choi et al., “An Altered Pattern of Cross-Resistance in Multi-Drug-Resistant Human Cells Results From Spontaneous Mutations in the Mdr-1 (P-glycoprotein) Gene,” Cell, 53:519, 1988.

Cohen et al., “A Repetitive Sequence Element 3′ of the Human c-Ha-ras1 Gene Has Enhancer Activity,” J. Cell. Physiol., 5:75, 1987.

Costa et al., “The Cell-Specific Enhancer of the Mouse Transthyretin (Prealbumin) Gene Binds a Common Factor at One Site and a Liver-Specific Factor(s) at Two Other Sites,” Mol. Cell. Biol., 8:81, 1988.

Cox et al., J. Virol., 67:5664-5667, 1993.

Cripe et al., “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J, 6:3745, 1987.

Culotta and Hamer, “Fine Mapping of a Mouse Metallothionein Gene Metal-Response Element,” Mol. Cell. Biol., 9:1376, 1989.

Dandolo et al., “Regulation of Polyma Virus Transcription in Murine Embryonal Carcinoma Cells,” J. Virology, 47:55, 1983.

Davis et al., Hum. Molec. Genet., 2:1847-1851, 1993.

De Villiers et al., “Polyoma Virus DNA Replication Requires an Enhancer,” Nature, 312:242, 1984.

Deschamps et al., “Identification of a Transcriptional Enhancer Element Upstream From the Proto-Oncogene Fos,” Science, 230:1174, 1985.

Edbrooke et al., “Identification of cis-Acting Sequences Responsible for Phorbol Ester Induction of Human Serum Amyloid A Gene Expression Via a Nuclear-Factor-KB-like Transcription Factor,” Mol. Cell. Biol., 9:1908, 1989.

Edlund et al., “Cell-Specific Expression of the Rat Insulin Gene: Evidence for Role of Two Distinct 5′ Flanking Elements,” Science, 230:912, 1985.

Eisenbraun et al., DNA Cell. Biol., 12:791-797, 1993.

Eldridge et al., Adv. Exp. Med. Biol., 251:191-202, 1989.

Eldridge et al., Mol. Immunol., 228:287-293, 1991.

Feng and Holland, “HIV-I Tat Trans-Activation Requires the Loop Sequence Within Tar,” Nature, 334:6178, 1988.

Firak and Subramanian, “Minimal Transcription Enhancer of Simian Virus 40 is a 74-Base-Pair Sequence that Has Interacting Domains,” Mol. Cell. Biol., 6:3667, 1986.

Foecking and Hofstetter, “Powerful and Versatile Enhancer-Promoter Unit for Mammalian Expression Vectors,” Gene, 45:101, 1986.

Fujita et al., “Interferon-Beta Gene Regulation: Tandemly Repeated Sequences of a Synthetic 6-bp Oligomer Function as a Virus-Inducible Enhancer,” Cell, 49:357, 1987.

Fynan et al., Proc. Nat'l. Acad. Sci., USA, 90:11478-11482, 1993.

Gilles et al., “A Tissue-Specific Transcription Enhancer Element is Lcoated in the Major Intron of a Rearranged Immunoglobulin Heavy-Chain Gene,” Cell, 33:717, 1983.

Gloss et al., “The Upstream Regulatory Region of the Human Papilloma Virus-16 Contains an E2 Protein-Independent Enhancer Which is Specific for Cervical Carcinoma Cells and Regulated by Glucocorticoid Hormones,” EMBO J, 6:3735, 1987.

Godbout et al., “Fine-Structure Mapping of the Three Mouse Alpha-Fetoprotein Gene Enhancers,” Mol. Cell. Biol., 8:1169, 1988.

Goodbourn and Maniatis, “Overlapping Positive and Negative Regulatory Domains of the Human β-Interferon Gene,” Proc. Natl. Acad. Sci. USA, 85:1447, 1988.

Goodbourn et al., “The Human Beta-Interferon Gene Enhancer is Under Negative Control,” Cell, 45:601, 1986.

Greene et al., “HIV-1, and Normal T-Cell Growth: Transcriptional Strategies and Surprises,” Immunology Today, 10:272, 1989.

Grosschedl and Baltimore, “Cell-Type Specificity of Immunoglobulin Gene Expression is Regulated by at Least Three DNA Sequence Elements,” Cell, 41:885, 1985.

Haslinger and Karin, “Upstream Promoter Element of the Human Metallothionein-II Gene Can Act Like an Enhancer Element,” Proc. Natl. Acad. Sci. U.S.A., 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Hen et al., “A Mutated Polyoma Virus Enhancer Which is Active in Undifferentiated Embryonal Carcinoma Cells is not Repressed by Adenovirus-2 E1A Products,” Nature, 321:249, 1986.

Hensel et al., “PMA-Responsive 5′ Flanking Sequences of the Human TNF Gene,” Lymphokine Res., 8:347, 1989.

Herr and Clarke, “The SV40 Enhancer is Composed of Multiple Functional Elements That Can Compensate for One Another,” Cell, 45:461, 1986.

Hirochika et al., “Enhancers and Trans-Acting E2 Transcriptional Factors of Papilloma Viruses,” J. Virol., 61:2599, 1987.

Hirsch et al., “Identification of Positive and Negative Regulatory Elements Governing Cell-Type-Specific Expression of the Neural-Cell-Adhesion-Molecule Gene,” Mol. Cell. Biol., 10:1959,1990.

Holbrook et al., “cis-Acting Transcriptional Regulatory Sequences in the Gibbon Ape Leukemia Virus (GALV) Long Terminal Repeat,” Virology, 157:211, 1987.

Horlick and Benfield, “The Upstream Muscle-Specific Enhancer of the Rat Muscle Creatine Kinase Gene is Composed of Multiple Elements,” Mol. Cell. Biol., 9:2396, 1989.

Huang et al., “Glucocorticoid Regulation of the Ha-MuSV p21 Gene Conferred by Sequences From Mouse Mammary Tumor Virus,” Cell, 27:245, 1981.

Hwang et al., “Characterization of the S-Phase-Specific Transcription Regulatory Elements in a DNA-Replication-Independent Testis-Specific H2B (TH2B) Histone Gene,” Mol. Cell. Biol., 10:585, 1990.

Imagawa et al., “Transcription Factor AP-2 Mediates Induction by Two Different Signal-Transduction Pathways: Protein Kinase C and cAMP,” Cell, 51:251, 1987.

Imbra and Karin, “Phorbol Ester Induces the Transcriptional Stimulatory Activity of the SV40 Enhancer,” Nature, 323:555, 1986.

Imler et al., “Negative Regulation Contributes to Tissue Specificity of the Immunoglobulin Heavy-Chain Enhancer,” Mol. Cell. Biol., 7:2558, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875, 1984.

Inouye et al., “Up-promoter mutations in the 1 pp gene of Escherichia coli,” Nucl. Acids Res., 13:3101-3109, 1985.

Inouye et al., “Up-promoter mutations in the 1 pp gene of Escherichia coli,” Nucl. Acids Res., 13:3101-3109, 1985.

Jakobovits et al., “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555, 1988.

Jameel and Siddiqui, “The Human Hepatitis B Virus Enhancer Requires Transacting Cellular Factor(s) for Activity,” Mol. Cell. Biol., 6:710, 1986.

Jaynes et al., “The Muscle Creatine Kinase Gene is Regulated by Multiple Upstream Elements, Including a Muscle-Specific Enhancer,” Mol. Cell. Biol., 8:62, 1988.

Johnson et al., “Muscle Creatine Kinase Sequence Elements Regulating Skeletal and Cardiac Muscle Expression in Transgenic Mice,” Mol. Cell. Biol., 9:3393, 1989.

Johnston and Tang, Genetic Engineering, 15:225-236, 1993.

Kadesch and Berg, “Effects of the Position of the Simian Virus 40 Enhancer on Expression of Multiple Transcription Units in a Single Plasmid,” Mol. Cell. Biol., 6:2593, 1986.

Karin et al., “Metal-Responsive Elements Act as Positive Modulators of Human Metallothionein-IIA Enhancer Activity,” Mol. Cell. Biol., 7:606, 1987.

Katinka et al., “Expression of Polyoma Early Functions in Mouse Embryonal Carcinoma Cells Depends on Sequence Rearrangements in the Beginning of the Late Region,” Cell, 20:393, 1980.

Katinka et al., “Polyoma DNA Sequences Involved in the Control of Viral Gene Expression in Murine Embryonal Carcinoma Cells,” Nature, 290:720, 1981.

Kawamoto et al., “Identification of the Human Beta-Actin Enhancer and its Binding Factor,” Mol. Cell. Biol., 8:267, 1988.

Kiledjian et al., “Identification and Characterization of Two Functional Domains Within the Murine Heavy-Chain Enhancer,” Mol. Cell. Biol., 8:145, 1988.

Klamut et al., “Molecular and Functional Analysis of the Muscle-Specific Promoter Region of the Duchenne Muscular Dystrophy Gene,” Mol. Cell. Biol., 10:193, 1990.

Koch et al., “Anatomy of a New B-Cell-Specific Enhancer,” Mol. Cell. Biol., 9:303, 1989.

Krieg, et al., Nature, 374:546-549, 1995.

Kriegler and Botchan, “A Retrovirus LTR Contains a New Type of Eukaryotic Regulatory Element,” In: Eukaryotic Viral Vectors, ed. Y. Gluzman. Cold Spring Harbor: Cold Spring Harbor Laboratory, NY, 1982.

Kriegler and Botchan, “Enhanced transformation by a simian virus 40 recombinant virus containing a Harvey murine sarcoma virus long terminal repeat,” Mol. Cell. Biol. 3:325, 1983.

Kriegler et al., “A Novel Form of TNF/Cachectin Is a Cell-Surface Cytotoxix Transmembrane Protein: Ramifications for the Complex Physiology of TNF,” Cell, 53:45, 1988.

Kriegler et al., “Promoter Substitution and Enhancer Augmentation Increases the Penetrance of the SV40 A Gene to Levels Comparable to That of the Harvey Murine Sarcoma Virus Ras Gene in Morphologic Transformation,” In: Gene Expression, eds. D. Hamer and M. Rosenberg. New York: Alan R. Liss, 1983.

Kriegler et al., “Transformation Mediated by the SV40 T Antigens: Separation of the Overlapping SV40 Early Genes with a Retroviral Vector,” Cell, 38:483, 1984a.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. eds, Cold Spring Harbor: Cold Spring Harbor Laboratory, 1984b.

Kuhl et al., “Reversible Silencing of Enhancers by Sequences Derived From the Human IFN-alpha Promoter,” Cell, 50:1057, 1987.

Kunz et al., “Identification of the Promoter Sequences Involved in the Interleukin-6-Dependent Expression of the Rat Alpha-2-Macroglobulin Gene,” Nuc. Acids Res., 17:1121, 1989.

Larsen et al., “Repression Medaites Cell-Type-Specific Expression of the Rat Growth Hormone Gene,” Proc. Natl. Acad. Sci. U.S.A., 83:8283, 1986.

Laspia et al., “HIV-1 Tat Protein Increases Transcriptional Initiation and Stabilizes Elongation,” Cell, 59:283, 1989.

Latimer et al., “Highly Conserved Upstream Regions of the α₁-Antitrypsin Gene in Two Mouse Species Govern Liver-Specific Expression by Different Mechanisms,” Mol. Cell. Biol., 10:760, 1990.

Lee et al., “Glucocorticoids Regulate Expression of Dihydrofolate Reductase cDNA in Mouse Mammary Tumor Virus Chimaeric Plasmids,” Nature, 294:228, 1981.

Levinson et al, “Activation of SV40 Genome by 72-Base-Pair Tandem Repeats of Moloney Sarcoma Virus,” Nature, 295:79, 1982.

Lin et al., “Delineation of an Enhancerlike Positive Regulatory Element in the Interleukin-2 Receptor α-Chain Gene,” Mol. Cell. Biol., 10:850, 1990.

Lowrie et al., Vaccine, 12:1537-1540, 1994.

Luria et al., “Promoter Ehancer Elements in the Rearranged Alpha-Chain Gene of the Human T-Cell Receptor,” EMBO J, 6:3307, 1987.

Lusky and Botchan, “Transient Replication of Bovine Papilloma Virus Type 1 Plasmids: cis and trans Requirements,” Proc. Natl. Acad. Sci. U.S.A., 83:3609, 1986.

Lusky et al., “Bovine Papilloma Virus Contains an Activator of Gene Expression at the Distal End of the Early Transcription Unit,” Mol. Cell. Biol. 3:1108, 1983.

Majors and Varmus, “A Small Region of the Mouse Mammary Tumor Virus Long Terminal Repeat Confers Glucocorticoid Hormone Regulation on a Linked Heterologous Gene,” Proc. Natl. Acad. Sci. U.S.A., 80:5866, 1983.

Manicken et al., J. Immunol., 155:259-265, 1995.

Marx et al., Science, 260:1323-1328, 1993.

McNeall et al., “Hyperinducible Gene Expression From a Metallotionein Promoter Containing Additional Metal-Responsive Elements,” Gene, 76:81, 1989.

Miksicek et al., “Glucocorticoid Responsiveness of the Transcriptional Enhancer of Moloney Murine Sarcoma Virus,” Cell, 46:203, 1986.

Montgomery et al., DNA Cell. Biol., 12:777-783, 1993.

Mordacq and Linzer, “Co-localization of Elements Required for Phorbol Ester Stimulation and Glucocorticoid Repression of Proliferin Gene Expression,” Genes and Dev., 3:760, 1989.

Moreau et al., “The SV40 Base-Repair Repeat Has a Striking Effect on Gene Expression Both in SV40 and Other Chimeric Recombinants,” Nuc. Acids Res., 9:6047, 1981.

Musesing et al., “Regulation of mRNA Accumulation by a Human Immunodeficiency Virus Trans-Activator Protein,” Cell, 48:691, 1987.

Nabel et al., Hum. Gene Ther., 3:649-656, 1992.

Ng et al., “Regulation of the Human Beta-Actin Promoter by Upstream and Intron Domains,” Nuc. Acids Res., 17:601, 1989.

O'Hagan et al., Immunology, 73:239-242, 1991.

O'Hagen et al., Vaccine, 11:149-154, 1993.

Ondek et al, “Discrete Elements Within the SV40 Enhancer Region Display Different Cell-Specific Enhancer Activities,” EMBO J., 6:1017, 1987.

Ornitz et al., “Promoter and Enhancer Elements From the Rat Elastase I Gene Function Independently of Each Other and of Heterologous Enhancers,” Mol. Cell. Biol., 7:3466, 1987.

Palmiter et al, “Differential Regulation of Metallothionein-Thymidine Kinase Fusion Genes in Transgenic Mice and Their Offspring,” Cell, 29:701, 1982.

Pech et al., “Functional Identification of Regulatory Elements Within the Promoter Region of Platelet-Derived Growth Factor 2,” Mol. Cell. Biol., 9:396, 1989.

Pecorino and Lo, Current Biol., 2:30-32, 1992.

Perez-Stable and Constantini, “Roles of Fetal γ-globin Promoter Elements and the Adult β-globin 3′ Enhancer in the Stage-Specific Expression of Globin Genes,” Mol. Cell. Biol., 10:1116, 1990.

Picard and Schaffner, “A Lymphocyte-Specific Enhancer in the Mouse Immunoglobulin Kappa Gene,” Nature, 307:83, 1984.

Pinkert et al., “An Albumin Enhancer Located 10 kb Upstream Functions Along With its Promoter to Direct Efficient, Liver-Specific Expression in Transgenic Mice,” Genes and Dev., 1:268, 1987.

Ponta et al., “Hormonal Response Region in the Mouse Mammary Tumor Virus Long Terminal Repeat Can Be Dissociated From the Proviral Pomoter and Has Enhancer Properties,” Proc. Natl. Acad. Sci. U.S.A., 82:1020, 1985.

Porton et al., “Immunoglobulin Heavy-Chain Enhancer is Required to Maintain Transfected γ2A Gene Expression in a pre-B-cell Line,” Mol Cell. Biol., 10: 1076, 1990.

Queen and Baltimore, “Immunoglobulin Gene Transcription is Activated by Downstream Sequence Elements,” Cell, 35:741, 1983.

Quinn et al., “Multiple Components are Required for Sequence Recognition of the AP1 Site in the Gibbon Ape Leukemia Virus Enhancer,” Mol. Cell. Biol., 9:4713, 1989.

Redondo et al., “A T-Cell-Specific Transcriptional Enhancer Within the Human T-Cell Receptor δ Locus,” Science, 247:1225, 1990.

Reisman and Rotter, “Induced Expression From the Moloney Murine Leukemia Virus Long Terminal Repeat During Differentiation of Human Myeloid Cells is Mediated Through its Transcriptional Enhancer,” Mol. Cell. Biol., 9:3571, 1989.

Resendez Jr. et al., “Identification of Highly Conserved Regulatory Domains and Protein-Binding Sites in the Promoters of the Rat and Human Genes Encoding the Stress-Inducible 78-kilodalton Glucose-Regulated Protein,” Mol. Cell. Biol., 8:4579, 1988.

Ripe et al, “Regulatory Elements in the 5′ Flanking Region and the First Intron Contribute to Transcriptional Control of the Mouse alpha-1-type Collagen Gene,” Mol. Cell. Biol., 9:2224,1989.

Rittling et al., “AP-1/jun-binding Sites Mediate Serum Inducibility of the Human Vimentin Promoter,” Nuc. Acids Res., 17:1619, 1989.

Robinson et al., Vaccine, 11:957-960, 1993.

Rosen et al., “The Location of cis-acting Regulatory Sequences in the Human T-Cell Lymphotropic Virus Type III (HTLV-111/LAV) Long Terminal Repeat,” Cell, 41:813, 1988.

Rousse et al., J. Virol., 68:5685-5689, 1994.

Sakai et al. , “Hormone-Mediated Repression: A Negative Glucocorticoid-Response Element From the Bovine Prolactin Gene,” Genes and Dev., 2:1144, 1988.

Sambrook, Fritsch, Maniatis, In: Molecular Cloning: A Laboratory Manual, Vol. 1, Cold Spring Harbor Laboratory Press, Cold Spring Harbor, N.Y., Ch. 7,7.19-17.29, 1989.

Sambrook, Fritsch, Maniatis, In: Molecular Cloning: A Laboratory Manual, Vol. 1, Cold Spring Harbor Laboratory Press, Cold Spring Harbor, N.Y., Ch. 7,7.19-17.29, 1989.

Satake et al., “Biological Activities of Oligonucleotides Spanning the F9 Point Mutation Within the Enhancer Region of Polyoma Virus DNA,” J. Virology, 62:970, 1988.

Schaffner et al., “Redundancy of Information in Enhancers as a Principle of Mammalian Transcription Control,” J. Mol. Biol., 201:81, 1988.

Searle et al., “Building a Metal-Responsive Promoter With Synthetic Regulatory Elements,” Mol. Cell. Biol., 5:1480, 1985.

Sedagah et al., Proc. Nat'l Acad. Sci., USA, 91:9866-9870, 1994.

Sharp and Marciniak, “HIV Tar: an RNA Enhancer?,” Cell, 59:229, 1989.

Shaul and Ben-Levy, “Multiple Nuclear Proteins in Liver Cells are Bound to Hepatitis B Virus Enhancer Element and its Upstream Sequences,” EMBO J, 6:1913, 1987.

Sherman et al., “Class II Box Consensus Sequences in the HLA-DRα Gene: Transcriptional Function and Interaction with Nuclear Proteins,” Mol. Cell. Biol., 9:50, 1989.

Sleigh and Lockett, “SV40 Enhancer Activation During Retinoic-Acid-Induced Differentiation of F9 Embryonal Carcinoma Cells,” J. EMBO, 4:3831, 1985.

Spalholz et al., “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J., 2:1193, 1983.

Stephens and Hentschel, “The Bovine Papilloma Virus Genome and its Uses as a Eukaryotic Vector,” Biochem. J., 248:1, 1987.

Stuart et al., “Identification of Multiple Metal Regulatory Elements in Mouse Metallothionein-I Promoter by Assaying Synthetic Sequences,” Nature, 317:828, 1985.

Sullivan and Peterlin, “Transcriptional Enhancers in the HLA-DQ Subregion,” Mol. Cell. Biol., 7:3315, 1987.

Swartzendruber and Lehman, “Neoplastic Differentiation: Interaction of Simian Virus 40 and Polyoma Virus with Murine Teratocarcinoma Cells,” J. Cell. Physiology, 85:179, 1975.

Takebe et al., “SRα Promoter: An Efficient and Versatile Mammalian cDNA Expression System Composed of the Simian Virus 40 Early Promoter and the R-U5 Segment of Human T-Cell Leukemia Virus Type 1 Long Terminal Repeat,” Mol. Cell. Biol., 8:466, 1988.

Tang et al., Nature, 356:152-154, 1992.

Tavernier et al., “Deletion Mapping of the Inducible Promoter of Human IFN-beta Gene,” Nature, 301:634, 1983.

Taylor and Kingston, “E1a Trans-Activation of Human HSP70 Gene Promoter Substitution Mutants is Independent of the Composition of Upstream and TATA Elements,” Mol. Cell. Biol., 10:176, 1990b.

Taylor and Kingston, “Factor Substitution in a Human HSP70 Gene Promoter: TATA-Dependent and TATA-Independent Interactions,” Mol. Cell. Biol., 10:165, 1990a.

Taylor et al., “Stimulation of the Human Heat-Shock Protein 70 Promoter in vitro by Simian Virus 40 Large T Antigen,” J. Biol. Chem., 264:15160, 1989.

Thiesen et al., “A DNA Element Responsible for the Different Tissue Specificities of Friend and Moloney Retroviral Enhancers,” J. Virology, 62:614.

Treisman, “Transient Accumulation of c-fos RNA Following Serum Stimulation Requires a Conserved 5′ Element and c-fos 3′ Sequences,” Cell, 42:889, 1985.

Tronche et al., “Anatomy of the Rat Albumin Promoter,” Mol. Biol. Med., 7:173, 1990.

Tronche et al., “The Rat Albumin Promoter: Cooperation with Upstream Elements is Required When Binding of APF/HNF 1 to the Proxinal Element is Partially Impaired by Mutation or Bacterial Methylation,” Mol. Cell. Biol., 9:4759, 1989.

Trudel and Constantini, “A 3′ Enhancer Contributes to the Stage-Specific Expression of the Human Beta-Globin Gene,” Genes and Dev., 6:954, 1987.

Tyndall et al., “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,682,195

U.S. Pat. No. 4,683,202

Ulmer et al., Science, 259:1745-1749, 1993.

Vannice and Levinson, “Properties of the Human Hepatitis B Virus Enhancer: Position Effects and Cell-Type Nonspecificity,” J. Virology, 62:1305, 1988.

Vasseur et al., “Isolation and Characterization of Polyoma Virus Mutants Able to Develop in Multipotential Murine Embryonal Carcinoma Cells,” Proc. Natl. Acad. Sci. U.S.A., 77:1068, 1980.

Wang and Calame, “SV40 Enhancer-Binding Factors are Required at the Establishment but not the Maintenance Step of Enhancer-Dependent Transcriptional Activation,” Cell, 47:241, 1986.

Wang et al., 1993.

Weber et al., “An SV40 ‘Enhancer Trap’ Incorporates Exogenous Enhancers or Generates Enhancers From its Own Sequences,” Cell, 36:983, 1984.

Weinberger et al. “Localization of a Repressive Sequence Contributing to B-cell Specificity in the Immunoglobulin Heavy-Chain Enhancer,” Mol. Cell. Biol., 8:988, 1984.

Williams et al., Proc Natl Acad Sci USA , 88:2726-2730, 1991

Winoto and Baltimore, “αβ-lineage-specific Expression of the α T-Cell Receptor Gene by Nearby Silencers,” Cell, 59:649, 1989.

Wolff et al., Science, 247:1465-1468, 1990.

Xiang et al., Virology, 199:132-140, 1994.

Yokoyama et al., J. Virol., 6964:2684-2688, 1995.

Yoshimura et al., Nucleic Acids Reg., 12:3233-3240, 1992

Yutzey et al. “An Internal Regulatory Element Controls Troponin I Gene Expression,” Mol. Cell. Biol., 9:1397, 1989.

What is claimed is:

1. A composition for inducing an immune response in an animal, comprising:
a) an expression vector containing a DNA sequence encoding at least one immunogenic polypeptide, said expression vector further encoding an expression control sequence, under the control of a regulatory element, capable of directing expression said immunogenic polypeptide;
b) at least one regulatory protein; and
c) at least one regulatory moiety.
wherein the expression of said immunogenic polypeptide is controlled by said regulatory protein wherein said regulatory protein interacts with said regulatory moiety and activates said regulatory element.

2. The composition of claim 1, wherein said regulatory protein is encoded by a second expression vector.

3. The composition of claim 1, wherein said regulatory moiety interacts with said regulatory protein and renders it active.

4. The composition of claim 3, wherein said activated regulatory protein interacts with said regulatory element of said expression vector and directs expression of said immunogenic polypeptide.

5. The composition of claim 1, wherein said regulatory moiety is a small molecule.

6. The composition of claim 1, wherein said regulatory moiety is a drug.

7. The composition of claim 1, wherein said regulatory moiety is administered orally.

8. The composition of claim 1, wherein said regulatory moiety is administered intravenously.

9. The composition of claim 1, wherein an adjuvant is administered in addition to said regulatory moiety.

10. The composition of claim 9, wherein said adjuvant is selected from a group consisting of alum, CpG oligonucleotides, guanosine tetraphosphate and guanosine pentaphosphate.

11. A method for inducing an immune response in an animal, comprising administering:
a) an expression vector containing a DNA sequence encoding at least one immunogenic polypeptide, said expression vector further encoding an expression control sequence, under the control of a regulatory element, capable of directing expression said immunogenic polypeptide; and
b) at least one regulatory moiety.
wherein the expression of said immunogenic polypeptide is controlled by a regulatory protein wherein said regulatory protein interacts with said regulatory moiety.

12. The method of claim 11, wherein said regulatory protein is endogenously encoded in the animal.

13. The method of claim 11, further comprising administration of said regulatory protein.

14. The method of claim 13, wherein said regulatory protein is encoded by a second expression vector.

15. The method of claim 11, wherein said regulatory moiety interacts with said regulatory protein and renders it active.

16. The method of claim 11, wherein said activated regulatory protein interacts with said regulatory element of said expression vector and directs expression of said immunogenic polypeptide.

17. The method of claim 11, wherein said regulatory moiety is a small molecule.

18. The method of claim 11, wherein said regulatory moiety is a drug.

19. The method of claim 11, wherein said regulatory moiety is administered orally.

20. The method of claim 11, wherein said regulatory moiety is administered intravenously.

21. The method of claim 11, wherein an adjuvant is administered in addition to said regulatory moiety.

22. The method of claim 21, wherein said adjuvant is selected from a group consisting of alum, CpG oligonucleotides, guanosine tetraphosphate and guanosine pentaphosphate.

23. A method for producing antibodies, comprising administering:
a) an expression vector containing a DNA sequence encoding at least one immunogenic polypeptide, said expression vector further encoding an expression control sequence, under the control of a regulatory element, capable of directing expression said immunogenic polypeptide; and
b) at least one regulatory moiety.
wherein the expression of said immunogenic polypeptide is controlled by a regulatory protein wherein said regulatory protein interacts with said regulatory moiety.

24. The method of claim 23, wherein said regulatory protein is endogenously encoded in the animal.

25. The method of claim 23, further comprising administration of said regulatory protein.

26. The method of claim 25, wherein said regulatory protein is encoded by a second expression vector.

27. The method of claim 23, wherein said regulatory moiety interacts with said regulatory protein and renders it active.

28. The method of claim 23, wherein said activated regulatory protein interacts with said regulatory element of said expression vector and directs expression of said immunogenic polypeptide.

29. The method of claim 23, wherein said regulatory moiety is a small molecule.

30. The method of claim 23, wherein said regulatory moiety is a drug.

31. The method of claim 23, wherein said regulatory moiety is administered orally.

32. The method of claim 23, wherein said regulatory moiety is administered intravenously.

33. The method of claim 23, wherein an adjuvant is administered in addition to said regulatory moiety.

34. The method of claim 33, wherein said adjuvant is selected from a group consisting of alum, CpG oligonucleotides, guanosine tetraphosphate and guanosine pentaphosphate.

35. A method for producing an immunogenic polypeptide, comprising administering:
a) an expression vector containing a DNA sequence encoding at least one immunogenic polypeptide, said expression vector further encoding an expression control sequence, under the control of a regulatory element, capable of directing expression said immunogenic polypeptide; and
b) at least one regulatory moiety.
wherein the expression of said immunogenic polypeptide is controlled by a regulatory protein wherein said regulatory protein interacts with said regulatory moiety.

36. The method of claim 35, wherein said regulatory protein is endogenously encoded in the animal.

37. The method of claim 35, further comprising administration of said regulatory protein.

38. The method of claim 37, wherein said regulatory protein is encoded by a second expression vector.

39. The method of claim 35, wherein said regulatory moiety interacts with said regulatory protein and renders it active.

40. The method of claim 35, wherein said activated regulatory protein interacts with said regulatory element of said expression vector and directs expression of said immunogenic polypeptide.

41. The method of claim 35, wherein said regulatory moiety is a small molecule.

42. The method of claim 35, wherein said regulatory moiety is a drug.

43. The method of claim 35, wherein said regulatory moiety is administered orally.

44. The method of claim 35, wherein said regulatory moiety is administered intravenously.

45. The method of claim 35, wherein an adjuvant is administered in addition to said regulatory moiety.

46. The method of claim 45, wherein said adjuvant is selected from a group consisting of alum, CpG oligonucleotides, guanosine tetraphosphate and guanosine pentaphosphate.
